(19) United States
(12) Patent Application Publication
(10) Pub. No.: US 2021/0202058 A1
(43) Pub. Date: Jul. 1, 2021

(54) METHOD, MOBILE DEVICE AND STORAGE MEDIUM FOR VACCINE ADMINISTRATION REMINDING

(71) Applicant: COMPAL ELECTRONICS, INC., Taipei (TW)

(72) Inventors: HAO-CHEN WENG, Taipei (TW); CHIAO-TSU CHIANG, Taipei (TW); CHIEH-YU CHAN, Taipei (TW); YA-FANG HSU, Taipei (TW)

(21) Appl. No.: 17/012,083

(22) Filed: Sep. 4, 2020

Related U.S. Application Data
(60) Provisional application No. 62/953,912, filed on Dec. 26, 2019.

(51) Int. Cl. G16H 20/17; G16H 10/60

(52) U.S. Cl. CPC G16H 20/17; G16H 10/60

(57) ABSTRACT

A vaccine administration reminding method includes the steps of providing a vaccination reminder message to a user of a mobile device according to a vaccine administration receiver's birth date, displaying a list of potential hospitals according to a user behavior of the user on the mobile device, and recording a vaccination hospital selected by the user from the list. The user behavior may include browsing a web page of the vaccination hospital on the mobile device or dialing a telephone number of the vaccination hospital on the mobile device.

### CROSS-REFERENCE TO RELATED APPLICATION

This non-provisional application claims priority under 35 U.S.C. § 119(e) on U.S. provisional Patent Application No(s). 62/953,912 filed on Dec. 26, 2019, the entire contents of which are hereby incorporated by reference.

### BACKGROUND OF THE INVENTION

### 1. Field of the Invention

The present disclosure relates to a method for event reminding, and in particular to a vaccine administration reminding method according to a user behavior of a user on a mobile phone.

### 2. Description of the Related Art

Electronics engineers have tried for a long time to provide an electronic device capable of automatically reminding a user of forthcoming events. A common method is to provide a user interface for users to set personal work items in a calendar on the electronic device. When one of the work items is about due, the electronic device will sand a reminder message to the user. In another common way, multiple users collaborate over the cloud while one of the users is responsible for inputting the work items. When one of the work item is about due, the collaboration platform on the cloud automatically sends a reminder message to all the related users. This method has an advantage that not all the users have to set a reminder for such work item.

Under certain circumstances, a user of an electronic device needs to receive a reminder message that involves another person. However, it is possible the user is too busy to set the reminder while the other person is unable to set the reminder. As a result, the user might miss some important events. For instance, the parents usually need to take their children to hospital for routine vaccinations within a specified time period. However, it is possible the parents are too busy to set the vaccination date in the calendar on the electronic device and the children do not have the ability to do this. Therefore, it is desirable to develop an automatic vaccine administration reminding method.

### BRIEF SUMMARY OF THE INVENTION

It is an object of the present disclosure to provide a vaccine administration reminding method, which includes the steps of providing a vaccination reminder message to a user of a mobile device according to a birth date of a vaccination receiver; displaying a list of potential hospitals on the mobile device according to a user behavior of the user on the mobile device; and recording a vaccination hospital selected by the user from the list. The user behavior includes browsing a web page of the vaccination hospital on the mobile device, or dialing a phone number of the vaccination hospital on the mobile device.

In an embodiment of the present disclosure, the vaccination reminder message is provided further according to previous vaccine administration records of the vaccination receiver or a piece of external environment information. The vaccination hospital is recorded in a calendar on the mobile device.

In an embodiment, the user's arrival at the vaccination hospital can be determined according to a location of the mobile device or an image captured by the mobile device. After the user's arrival at the vaccination hospital, occurrence information of vaccination side effects previously input to the mobile device by the user and handling information corresponding to the occurrence information of vaccination side effects are displayed on the mobile device.

In an embodiment, it can be determined whether the user has left the vaccination hospital according to a location of the mobile device. After the user has left the vaccination hospital, occurrence information of vaccination side effects input to the mobile device by the user and handling information corresponding to the occurrence information of vaccination side effects are recorded in the mobile device.

To achieve at least the above object, the present disclosure also provides a mobile device, which includes a processor, a display unit and a storage unit. The storage unit has a plurality of program instructions stored therein. After executing the program instructions, the processor provides a vaccination reminder message to a user of the mobile device according to a birth date of a vaccination receiver, displays a list of potential hospitals on the mobile device according to a user behavior of the user on the mobile device, and records a vaccination hospital selected by the user from the list. The user behavior includes browsing a web page of the vaccination hospital on the mobile device, or dialing a phone number of the vaccination hospital on the mobile device.

To achieve at least the above object, the present disclosure also provides a non-transient storage medium having a plurality of program instructions stored therein. The program instructions are executable by a mobile device. After executing the program instructions, the mobile device provides a vaccination reminder message to a user of the mobile device according to a birth date of a vaccination receive, displays a list of potential hospitals on the mobile device according to a user behavior of the user on the mobile device, and records a vaccination hospital selected by the user from the list. The user behavior includes browsing a web page of the vaccination hospital on the mobile device, or dialing a phone number of the vaccination hospital on the mobile device.

With the above described technical means, the mobile device of the present disclosure can automatically provide a vaccine administration message related to a vaccination receiver (such as a child) to the user (such as the parent of the child) and can automatically record the vaccination hospital. Therefore, the present disclosure significantly avoids the user from missing a vaccination for a vaccination receiver.

### BRIEF DESCRIPTION OF THE DRAWINGS

FIG. 1 is a flowchart showing the steps of the vaccine administration reminding method according to an embodiment of the present disclosure.

FIG. 2 is a block diagram showing the structure of a mobile device for the vaccine administration reminding method according to a first embodiment of the present disclosure.

FIG. 3 is a block diagram showing the structure of a mobile device for the vaccine administration reminding method according to a second embodiment of the present disclosure.

FIG. 4 is a schematic view of a user interface for recording a vaccination hospital according to an embodiment of the present disclosure.

FIG. 5A is a schematic view of user interfaces for checking and inputting vaccination side effects as well as handling ways for the side effects.

FIG. 5B is a schematic view of user interfaces for checking and inputting vaccination side effects as well as handling ways for the side effects.

FIG. 5C is a schematic view of user interfaces for checking and inputting vaccination side effects as well as handling ways for the side effects.

FIG. 5D is a schematic view of user interfaces for checking and inputting vaccination side effects as well as handling ways for the side effects.

### DETAILED DESCRIPTION OF THE INVENTION

To facilitate understanding of the objects, characteristics and effects of this present disclosure, embodiments together with the attached drawings for the detailed description of the present disclosure are provided. It is noted the present disclosure can be implemented or applied in other embodiments, and many changes and modifications in the described embodiments can be carried out without departing from the spirit of the disclosure, and it is also understood that the preferred embodiments are only illustrative and not intended to limit the present disclosure in any way.

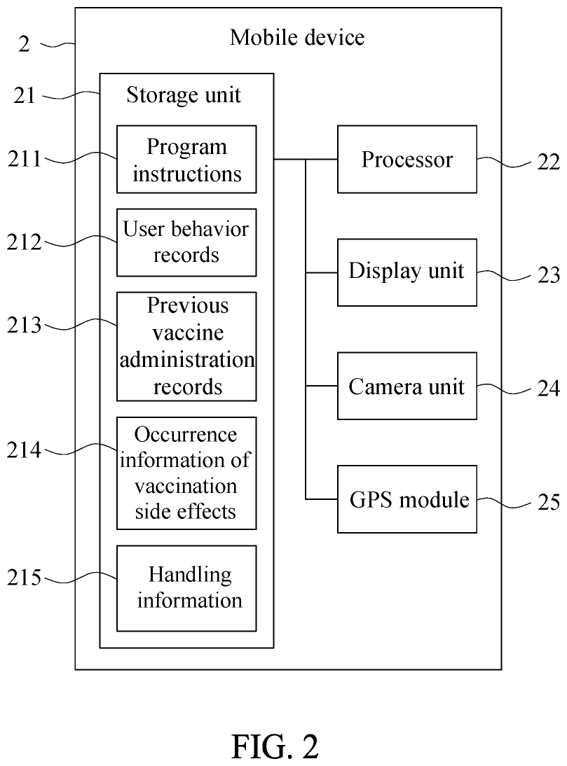

Please refer to FIG. 2. A mobile device 2 for a vaccine administration reminding method according to a first embodiment of the present disclosure can be a mobile phone or a tablet computer, which includes a storage unit 21, a processor 22, a display unit 23, a camera unit 24, and a global positioning system (GPS) module 25. The processor 22 can be a main processor of the mobile device 2. The display unit 23 can be a touch screen for showing user interfaces and converting user's touches into control signals. The storage unit 21 can be a non-transient memory for an electronic device, such as a flash memory or other types of memories. A plurality of program instructions 211 executable by the processor 22 are stored in the storage unit 21. After executing the program instructions 211, the processor 22 of the mobile device 2 can perform different steps corresponding to the program instructions 211.

In addition to the program instructions 211, the storage unit 21 also stores the user's user behavior records 212, previous vaccine administration records of the vaccination receivers 213, occurrence information of vaccination side effects 214, and handling information 215 for the vaccination side effects. The camera unit 24 captures images around the mobile device 2; and the captured images can also be stored in the storage unit 21. The GPS module 25 employs the global positioning system to obtain messages about the location of the mobile device 2.

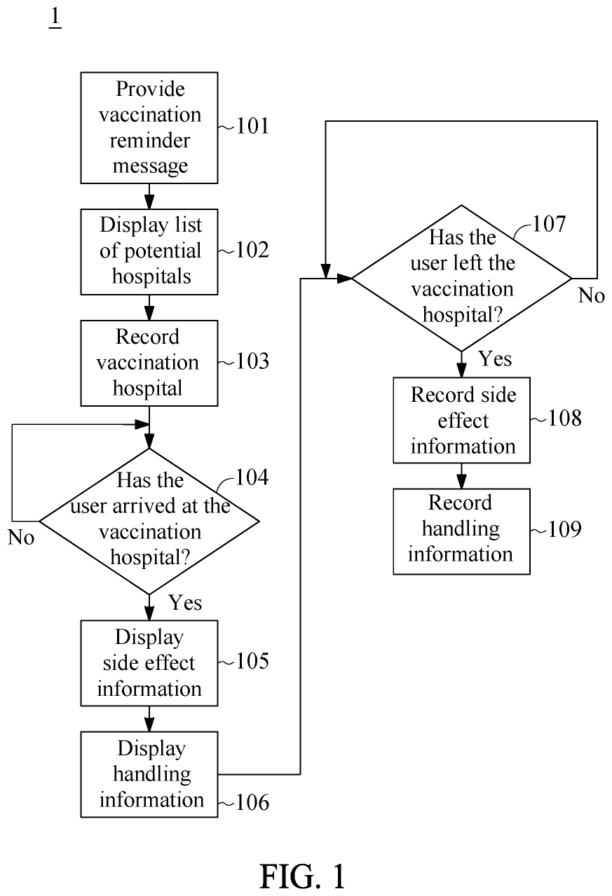

Please refer to FIG. 1 which shows a vaccine administration reminding flowchart 1 according to an embodiment of the present disclosure. As shown, after executing one of the program instructions 211, the processor 22 of the mobile device 2 would perform step 101, in which a vaccination reminder message is provided to a user according to a birth date of a vaccination receiver. The birth date of the vaccination receiver can be recorded in the storage unit 21 of the mobile device 2, or can be retrieved from cloud record. For instance, the user sets the vaccination receiver's birth date in the cloud health databank, and then sets the vaccination receiver's data stored in the cloud health databank which are accessible by the mobile device 2 from the Internet. With the settings, the mobile device 2 can obtain relevant information from the health databank from the Internet whenever the user needs to know the vaccination receiver's birth date. The step 101 may also be performed in other different manners. For instance, an external camera can be mounted in a house for monitoring a child's activities at home. The external camera can be mounted above a crib for monitoring a baby's activities. The images captured by the external camera are transmitted to a cloud server or the mobile device 2 for image recognition. If it is found the baby is not shown in the images while the signal of the GPS module 25 of the mobile device 2 indicates the user is not home, it is determined the baby might be outside with the user (or his/her parents). According to the baby's (i.e. the vaccination receiver's) birth date, the aforesaid image recognition and the aforesaid GPS module signal, a vaccination reminder message is either displayed on the mobile device 2 or provided to the mobile device 2 from the cloud server.

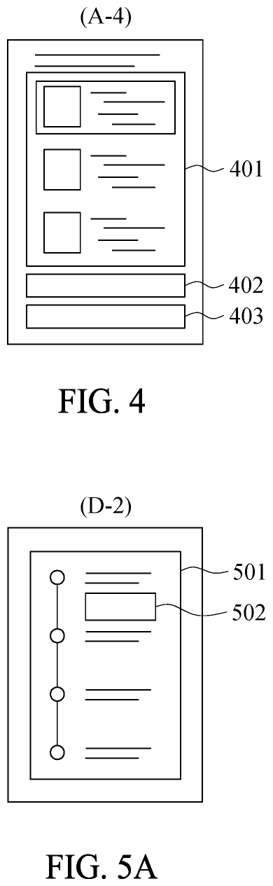

Then, the mobile device 2 performs step 102, in which the display unit 23 of the mobile device 2 displays a list 401 of potential hospitals according to the user behavior of the user on the mobile device 2. Please refer to FIG. 4. For example, if the user has browsed web pages or has dialed phone numbers on the mobile device 2, the processor 22 executing the program instruction 211 will analyze the web pages or the phone numbers and, for example, compares them with the web pages or phone numbers of the already known hospitals to determine whether the browsed web pages or the dialed phone numbers are hospital web pages or hospital phone numbers. If it is determined that the browsed web pages or the dialed phone numbers are hospital web pages or hospital phone numbers, the mobile device 2 will automatically compile information of the hospitals and display the list 401 of potential hospitals on the display unit 23 for the user. The user may directly touch the display unit 23 to select one of the hospitals as the vaccination hospital and clicks the option 402 below the list 401 to record the selected vaccination hospital. Or, the user may click the other option 403 to exit.

In the case that the user selects one hospital and clicks the option 402, the mobile device 2 performs the step 103 to record the vaccination hospital selected by the user from the list 401. For example, after the user has clicked the option 402, the mobile device 2 opens a calendar on the mobile device 2 and automatically adds the user selected vaccination hospital to the calendar as an event. The user may change the date, time or other related information of the vaccination event in the calendar.

Thereafter, the mobile device 2 performs step 104 to determine, according to a location of the mobile device 2, whether the user has already arrived at the vaccination hospital. The mobile device 2 can use the positioning information provided by the GPS module 25 to determine whether the user's location is within a specified range from the vaccination hospital. If the user's location is within a specified range from the vaccination hospital, it is determined the user has arrived at the vaccination hospital. Alternatively, the mobile device 2 can determine the user's position according to the images captured by the camera unit 24. For example, in the case that the user takes an image with the camera unit 24 and the hospital name is found in the image after an image recognition, it is determined the user has arrived at the vaccination hospital.

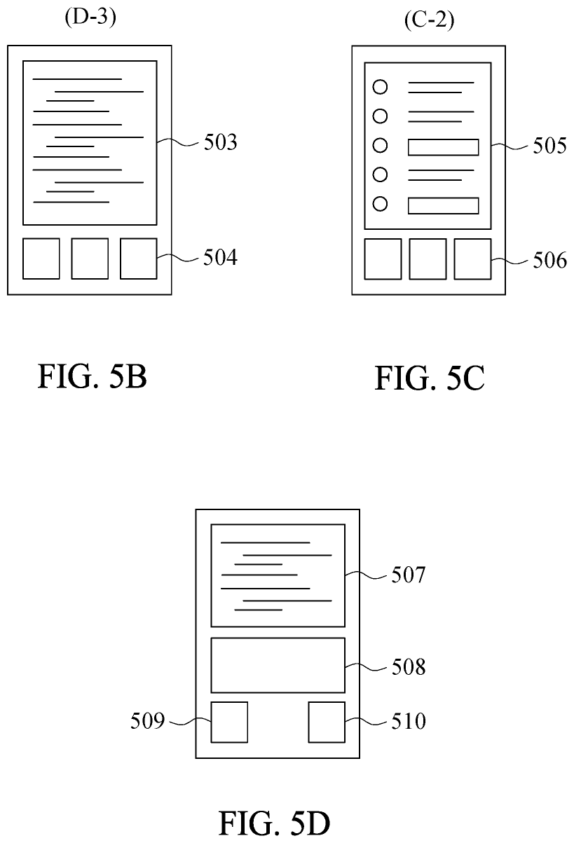

After the user has arrived at the vaccination hospital, the mobile device 2 performs steps 105 and 106 to display the occurrence information of vaccination side effects 214 and the corresponding handling information 215 previously input by the user. Please refer to FIGS. 5A and 5B. The mobile device 2 can display on the display unit 23 a vaccine administration status list 501, in which the vaccines that should be administered to the vaccination receiver are listed based on the vaccination receiver's birth date, and the vaccines that have been administered are marked. Below the vaccines that have been administered, a side effect information option 502 is shown for the user to click so as to show the occurrence information of side effects related to the administered vaccines. After the user has clicked the side effect information option 502, the mobile device 2 can show on the display unit 23 an interface, as shown in FIG. 5B, in which side effect information 502 previously input by the user and pictures 504 corresponding thereto are displayed. The side effect information 503 and the corresponding pictures 504 are the occurrence information of vaccination side effects 214 that was previously input by the user and stored in the storage unit 21.

Technically, it is advantageous that the program instructions 211 are executed to display the vaccine administration status list 501, the side effect information 503, and the pictures 504 after it is determined that the user has arrived at the vaccination hospital. For example, the user can be more efficiently reminded to provide the related information to the doctor or nurse for their information, and the user interface can be simpler and more convenient for operating. As a result, the user's burden can be reduced while the mobile device 2 is more convenient for use.

Then, the mobile device 2 performs step 107 to determine, according to a location of the mobile device 2, whether the user has left the vaccination hospital. Similar to the step 105, the mobile device 2 can use the positioning information from the GPS module 25 to determine whether the user's location is not within the specified range from the vaccination hospital. If the user's location is not within the specified range from the vaccination hospital, it is determined the user has left the vaccination hospital.

In the case that it is determined the user has left the vaccination hospital, the mobile device 2 performs the step 108 to provide an interface for the user to input the occurrence information of vaccination side effects 214. Please refer to FIG. 5C. The mobile device 2 displays a list 505 on the display unit 23 to show common side effects of the vaccines that has been administered to the vaccine receiver, so that the user can conveniently check and click on the list 505. The mobile device 2 also allows the user to upload pictures 506 that are captured using the camera unit 24 and have connection with the side effects, such as pictures showing skin rashes. The side effect information input by the user at the list 505, including the pictures 506 taken by the user, is recorded in the storage unit 21 of the mobile device 2 as the occurrence information of vaccine side effects 214.

In the case that occurrence information of vaccine side effects 214 is input by the user, the mobile device 2 performs the step 109 to record the handling information, which is input by the user and corresponding to the occurrence information of vaccine side effects 214. Please refer to FIG. 5D. The mobile device 2 displays available handling information 507 corresponding to the vaccine side effects and provides a text box for the user to input any handling status 508. The user may click on an option 509 to complete the inputting, or click on the option 510 to cancel the inputting. The handling status 508 input by the user is recorded in the storage unit 21 as the handling information 215 for future reference.

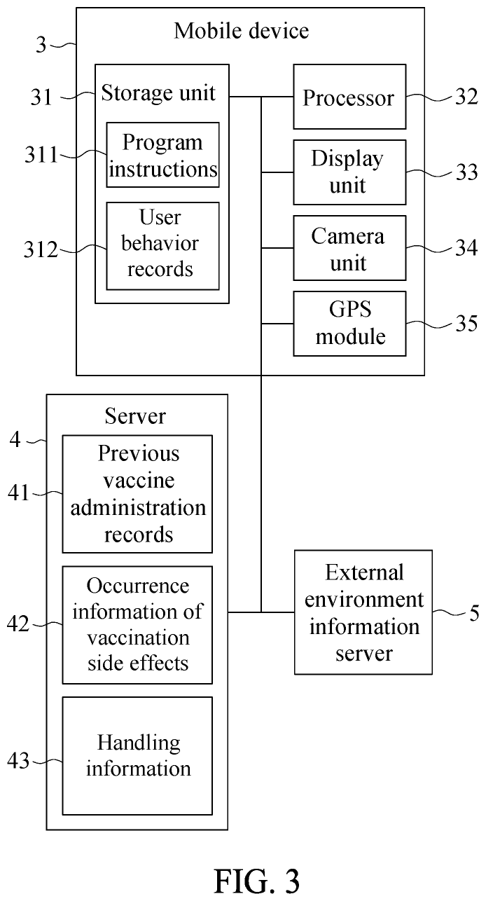

Various modifications can be made to the above embodiments by a person having ordinary skill in the art without departing from the scope of the present invention. For instance, please refer to FIG. 3 that shows a mobile device 3 according to a second embodiment of the present disclosure. In the second embodiment, the mobile device 3 includes a storage unit 31, a processor 32, a display unit 33, a camera unit 34, and a GPS module 35. Unlike the mobile device 2 in the first embodiment shown in FIG. 2, the storage unit 31 of the mobile device 3 stores only program instructions 311 and user behavior records 312. The previous vaccine administration records 41, occurrence information of vaccination side effects 42, and handling information 43 are stored in a server 4 on the Internet. The mobile device 3 may be connected to the server 4 in a wired or a wireless manner to access the above vaccination-related information. The server 4 can be a cloud health databank server.

Besides, the mobile device 3 can be connected to an external environment information server 5 to obtain external environment information, including infectious diseases that are spread currently. The mobile device 3 can perform step 101 to provide a vaccination reminder message to the user according to the external environment information. The external environment information server 5 can be, for example, an information server in a governmental agency. In the case that the governmental information server has published that it is currently the peak period of flu, the mobile device 3 provides a flu vaccine administration reminder message to the user according to the published information.

With the above described technical means, the mobile device of the present disclosure can automatically provide a vaccine administration message related to a vaccination receiver (such as a child) to the user (such as the parent of the child) and provide the user with a simple and user-friendly interface. Therefore, the present disclosure significantly avoids the user from missing a vaccination for a vaccination receiver.

While the present disclosure has been described by means of specific embodiments, numerous modifications and variations could be made thereto by those skilled in the art without departing from the scope and spirit of the present disclosure set forth in the claims.

What is claimed is:

1. A vaccine administration reminding method, comprising the steps of:
providing a vaccination reminder message to a user of a mobile device according to a birth date of a vaccination receiver;
displaying a list of potential hospitals on the mobile device according to a user behavior of the user on the mobile device; and
recording a vaccination hospital selected by the user from the list,
wherein the user behavior includes browsing a web page of the vaccination hospital on the mobile device, or dialing a phone number of the vaccination hospital on the mobile device.

2. The vaccine administration reminding method according to claim 1, wherein the vaccination reminder message is provided according to a vaccine administration record of the vaccination receiver.

3. The vaccine administration reminding method according to claim 2, wherein the vaccination reminder message is provided according to external environment information.

4. The vaccine administration reminding method according to claim 1, wherein the vaccination hospital is recorded in a calendar on the mobile device.

5. The vaccine administration reminding method according to claim 1, further comprising the step of:
determining whether the user has arrived at the vaccination hospital according to a location of the mobile device.

6. The vaccine administration reminding method according to claim 5, wherein arrival of the user at the vaccination hospital is further determined according to an image captured by the mobile device.

7. The vaccine administration reminding method according to claim 5, further comprising the step of:
displaying occurrence information of vaccination side effects input to the mobile device by the user.

8. The vaccine administration reminding method according to claim 7, further comprising the step of:
displaying handling information which corresponds to the occurrence information of the vaccination side effects and is input to the mobile device by the user.

9. The vaccine administration reminding method according to claim 1, further comprising the step of:
determining whether the user has left the vaccination hospital according to a location of the mobile device.

10. The vaccine administration reminding method according to claim 9, further comprising the step of:
recording occurrence information of vaccination side effects input to the mobile device by the user.

11. The vaccine administration reminding method according to claim 10, further comprising the step of:
recording handling information which corresponds to the occurrence information of vaccination side effects and is input to the mobile device by the user.

12. A mobile device, comprising:
a processor;
a display unit; and
a storage unit having a plurality of program instructions stored therein, wherein the processor performs the following steps after executing the program instructions:
providing a vaccination reminder message to a user of the mobile device according to a birth date of a vaccination receiver;
displaying a list of potential hospitals on the mobile device according to a user behavior of the user on the mobile device; and
recording a vaccination hospital selected by the user from the list;
wherein the user behavior includes browsing a web page of the vaccination hospital on the mobile device, or dialing a phone number of the vaccination hospital on the mobile device.

13. The mobile device according to claim 12, wherein the vaccination reminder message is provided according to a vaccine administration record of the vaccination receiver.

14. The mobile device according to claim 12, wherein the vaccination reminder message is provided according to external environment information.

15. The mobile device according to claim 12, wherein the vaccination hospital is recorded in a calendar on the mobile device.

16. The mobile device according to claim 12, wherein the processor further performs the following step after executing the program instructions:
determining whether the user has arrived at the vaccination hospital according to a location of the mobile device.

17. The mobile device according to claim 16, wherein arrival of the user at the vaccination hospital is further determined according to an image captured by the mobile device.

18. The mobile device according to claim 16, wherein the processor further performs the following step after executing the program instructions:
displaying occurrence information of vaccination side effects input to the mobile device by the user.

19. The mobile device according to claim 18, wherein the processor further performs the following step after executing the program instructions:
displaying handling information which corresponds to the occurrence information of vaccination side effects and is input to the mobile device by the user.

20. The mobile device according to claim 12, wherein the processor further performs the following step after executing the program instructions:
determining whether the user has left the vaccination hospital according to a location of the mobile device.

21. The mobile device according to claim 20, wherein the processor further performs the following step after executing the program instructions:
recording the occurrence information of vaccination side effects input to the mobile device by the user.

22. The mobile device according to claim 21, wherein the processor further performs the following step after executing the program instructions:
recording handling information which corresponds to the occurrence information of vaccination side effects and is input to the mobile device by the user.

23. A non-transient storage medium having a plurality of program instructions stored therein, and the program instructions being executable by a mobile device to perform the following steps:
providing a vaccination reminder message to a user of a mobile device according to a birth date of a vaccination receiver;
displaying a list of potential hospitals on the mobile device according to a user behavior of the user on the mobile device; and
recording a vaccination hospital selected by the user from the list,
wherein the user behavior includes browsing a webpage of the vaccination hospital on the mobile device, or dialing a phone number of the vaccination hospital on the mobile device.

24. The non-transient storage medium according to claim 23, wherein the vaccination reminder message is provided according to a vaccine administration record of the vaccination receiver.

25. The non-transient storage medium according to claim 23, wherein the vaccination reminder message is provided according to external environment information.

26. The non-transient storage medium according to claim 23, wherein the vaccination hospital is recorded in a calendar on the mobile device.

27. The non-transient storage medium according to claim 23, wherein the mobile device further performs the following step after executing the program instructions:
determining whether the user has arrived at the vaccination hospital according to a location of the mobile device.

28. The non-transient storage medium according to claim 27, wherein arrival of the user at the vaccination hospital is further determined according to an image captured by the mobile device.

29. The non-transient storage medium according to claim 27, wherein the mobile device further performs the following step after executing the program instructions:
displaying occurrence information of vaccination side effects input to the mobile device by the user.

30. The non-transient storage medium according to claim 29, wherein the mobile device further performs the following step after executing the program instructions:
displaying handling information which corresponds to the occurrence information of vaccination side effects and is input to the mobile device by the user.

31. The non-transient storage medium according to claim 23, wherein the mobile device further performs the following step after executing the program instructions:
determining whether the user has left the vaccination hospital according to a location of the mobile device.

32. The non-transient storage medium according to claim 31, wherein the mobile device further performs the following step after executing the program instructions:
recording occurrence information of vaccination side effects input to the mobile device by the user.

33. The non-transient storage medium according to claim 32, wherein the mobile device further performs the following step after executing the program instructions:
recording handling information which corresponds to the occurrence information of vaccination side effects and is input to the mobile device by the user.
